(19) United States
(12) Patent Application Publication
(10) Pub. No.: US 2014/0315322 A1
(43) Pub. Date: Oct. 23, 2014

(54) METHOD FOR MEASURING ACETIC ACID CONCENTRATION IN BLOOD PLASMA

(71) Applicant: FUSO PHARMACEUTICAL INDUSTRIES, LTD., Osaka-shi, Osaka (JP)

(72) Inventors: Shigeaki Yagi, Osaka-shi (JP); Manabu Nishizawa, Osaka-shi (JP); Hidetoshi Matsuzawa, Osaka-shi (JP); Hideo Nagase, Osaka-shi (JP)

(73) Assignee: FUSO PHARMACEUTICAL INDUSTRIES, LTD., Osaka-shi, Osaka (JP)

(21) Appl. No.: 14/367,047

(22) PCT Filed: Dec. 26, 2012
(86) PCT No.: PCT/JP2012/083663

§ 371 Date: Jun. 19, 2014

(30) Foreign Application Priority Data
Dec. 28, 2011 (JP) 2011-287835

(51) Int. Cl. G01N 30/72; G01N 33/50

(52) U.S. Cl. CPC G01N 30/7206; G01N 33/50
USPC 436/129; 250/282

(57) ABSTRACT

The present invention relates to providing a simple and highly reproducible method for measuring the concentration of acetic acid in blood plasma by using a gas chromatography/mass spectrometry (GC/MS), and more specifically relates to a method for measuring the concentration of acetic acid in blood plasma by using a gas chromatography/mass spectrometry (GC/MS), which comprises extracting acetic acid in blood plasma with methyl-tert-butyl ether (MTBE).

### TECHNICAL FIELD

The present application is filed claiming the priority of the Japanese Patent Application No. 2011-287835 (Filing date: Dec. 28, 2011), the entire contents of which are herein incorporated by reference.

The present invention relates to a simple and highly reproducible method for measuring the concentration of acetic acid in blood plasma (particularly, human blood plasma) by using a gas chromatography/mass spectrometry (GC/MS method).

### BACKGROUND ART

Among blood purification methods applicable to chronic renal failure patients and the like, the most common one is hemodialysis treatment. Hemodialysis treatment is used for removing waste products or water in blood, and correcting the concentration of serum electrolytes, correcting acid-base balance, etc. by dialysis agents.

Conventionally, acetate dialysis using acetate as an alkaline agent in dialysis fluid has been used. Nowadays, however, bicarbonate dialysis using sodium hydrogen carbonate as an alkaline agent is mainly used because it has been found that acetic acid exerts action on vasodilatation or inhibitory heart function. In bicarbonate dialysis, 8 to 12 mEq/L of acetic acid is still used as a pH regulator. A sharp rise in the concentration of acetic acid in blood plasma has been reported to lead to severe allergic reaction (Non-Patent Document 1). Accordingly, it is very important to measure the concentration of acetic acid in blood plasma in a patient during dialysis treatment in order to recognize its variation. As of now, however, the concentration of acetic acid in blood plasma is not examined in medical institutions or contract research organizations in Japan.

As methods for measuring the concentration of acetic acid in blood plasma, enzyme methods (Non-Patent Document 2 to 3), HPLC methods (Non-Patent Documents 4 to 6), GC methods (Non-Patent Documents 7 to 9) and GC/MS methods (Non-Patent Documents 10 to 12) have been reported. Among them, GC/MS methods have the highest specificity and sensitivity.

In conventional measurement methods using GC/MS, there is a problem of complicated procedures as follows: the results tend to vary depending on the operation due to using no stable isotope labeled acetic acid as an internal standard (Non-Patent Documents 10 and 12); it is necessary to add hydrochloric acid before extraction; diethyl ether having a low boiling point is used as an extracting solvent; and the like (Non-Patent Documents 11 and 12).

### PRIOR ART DOCUMENTS

### Non-Patent Documents

Non-Patent Document 1: Mamiko Ashizawa, et al.: Kyushu jinko toseki kenkyukai kaishi, 26, 87, (1998)
Non-Patent Document 2: Bergmeyer H U, In: Methods of Enzymatic Analysis, 3rd ed, vol VI, p 628-639
Non-Patent Document 3: Bartelt U and Kattermann R: J Clin. Chem. Clin. Biochem. 23: 879-881, 1985.
Non-Patent Document 4: Yamamoto M, et al.: Rinshoukensa 35(8): 977-880, 1991.
Non-Patent Document 5: Otake K, et al.: HDFryouhou: 266-269, 2008.
Non-Patent Document 6: Stein J, et al.: J. Chromatogr. 576: 53-61, 1992.
Non-Patent Document 7: Tollinger C D, et al.: Clin. Chem. 25/10: 1787-1790, 1979.
Non-Patent Document 8: Brazier M, et al.: Clin. Chim. Acta 148: 261-265 1985.
Non-Patent Document 9: Murase M, et al.: J. Chromatogr. B 664: 415-420 1995.
Non-Patent Document 10: Roccchiccioli F, et al.: Bio. and Enviro. Mass Sepctrom. 18: 816-819 1989.
Non-Patent Document 11: Pouteau E, et al.: J. Mass Spectrom. 36: 798-805 2001.
Non-Patent Document 12: Moreau N M, et al.: J. Chromatogr. B 784: 395-403 2003.

### SUMMARY OF INVENTION

### Technical Problem

An object of the present invention is to provide a simple and highly reproducible method for measuring the concentration of acetic acid in blood plasma by using a GC/MS method.

### Solution to Problem

The present inventors have studied so as to resolve the above problem and found that the number of operating procedures can be reduced by deproteinization and then liquid-liquid extraction directly without centrifugation after the deproteinization, and the operating procedures can be facilitated without the decrease of extraction efficiency by using, as an extracting solvent, methyl-tert-butyl ether (MTBE) that has a high boiling point, that is easy of handling, and that shows high extraction efficiency, thus leading to the present invention.

The present invention includes the followings:

[1] A method for measuring the concentration of acetic acid in blood plasma by using a gas chromatography/mass spectrometry (GC/MS method), which comprises extracting acetic acid in blood plasma with methyl-tert-butyl ether (MTBE).
[2] The method according to the above [1], which does not comprise adding hydrochloric acid before extracting.
[3] The method according to the above [1] or [2], which further comprises treating blood plasma with a deproteinizing agent.
[4] The method according to any one of the above [1] to [3], wherein the deproteinizing agent includes sulfosalicylic acid.
[5] The method according to the above [3] or [4], which comprises extracting acetic acid in blood plasma with MTBE, after treating blood plasma with a deproteinizing agent and not centrifuging.
[6] The method according to any one of the above [1] to [5], which further comprises adding a stable isotope labeled acetic acid to blood plasma.
[7] The method according to the above [6], wherein the stable isotope labeled acetic acid is selected from sodium acetate-1-¹³C, sodium acetate-2-¹³C, sodium acetate-¹³C₂, sodium acetate-d₃, sodium acetate-¹⁸O₂, sodium acetate-1-¹³C,d₃ and sodium acetate-2-¹³C,d₃.
[8] The method according to any one of the above [1] to [7], wherein the GC/MS method includes electron impact ionization.
[9] A kit for measuring the concentration of acetic acid in blood plasma by using a GC/MS method, comprising MTBE.
[10] The kit according to the above [9], which further comprises a deproteinizing agent.
[11] The kit according to the above [10], wherein the deproteinizing agent includes sulfosalicylic acid.
[12] The kit according to any one of the above [9] to [11], which further comprises a stable isotope labeled acetic acid.
[13] The kit according to the above [12], wherein the stable isotope labeled acetic acid is selected from sodium acetate-1-¹³C, sodium acetate-2-¹³C, sodium acetate-¹³C₂, sodium acetate-d₃, sodium acetate-¹⁸O₂, sodium acetate-1-¹³C,d₃ and sodium acetate-2-¹³C,d₃.
[14] The kit according to any one of the above [9] to [13], which further comprises a derivatizing reagent.
[15] The kit according to the above [14], wherein the derivatizing reagent is N-methyl-N-(tert-butyldimethylsilyl)trifluoroacetamide.

### Effects of Invention

According to the present invention, it is possible to measure the concentration of acetic acid in blood plasma in a simple and highly reproducible method due to the improvement of extraction efficiency of acetic acid by using MTBE having a high boiling point as an extracting solvent.

Further, according to the present invention, it is possible to perform solvent extraction directly, without centrifugation after the deprotainization and without addition of hydrochloric acid before the extraction.

### BRIEF DESCRIPTION OF DRAWINGS

FIG. 1 shows Selected Ion Monitoring (SIM) chromatograms of acetic acid and an internal standard of a sample for calibration curve. Acetic acid and the internal standard were separated and eluted in the vicinity of 7.45 and 7.41 min, respectively. The symbols in FIG. 1 refer to the followings: (a) acetic acid, (b) internal standard

FIG. 2 shows SIM chromatograms comparing human blood plasma with a sample for calibration curve (20 μmol/L). There was no interference peak derived from blood plasma at the elution position of the internal standard. The symbols in FIG. 2 refer to the followings: (a) acetic acid, (b) internal standard.

FIG. 3 shows a calibration curve obtained by a GC/MS method. In the range of concentration of 20 to 1000 μmol/L, the linearity of the calibration curve showed 0.999 of correlation coefficient (r), and the relative error (% RE) of each concentration of the calibration curve was from −5.4 to 7.5%.

FIG. 4 shows a calibration curve obtained by an enzyme method.

FIG. 5 shows a plot where the abscissa axis is the measured values obtained by the GC/MS method and the vertical axis is the measured values obtained by the enzyme method. There was a good correlation (r=0.9922) between the measured values obtained by both methods.

FIG. 6 shows a plot where the abscissa axis is the values obtained by the enzyme method relative to the values obtained by the GC/MS method as 100. Both values tended to be closer with increasing the values obtained by the GC/MS method.

### DESCRIPTION OF EMBODIMENTS

The present invention relates to a method for measuring the concentration of acetic acid in blood plasma by using a gas chromatography/mass spectrometry (GC/MS method), which comprises extracting acetic acid in blood plasma with methyl-tert-butyl ether (MTBE) (hereinafter sometimes referred to as “the method of the present invention”).

Blood plasma is one of liquid components of blood, accounts for 55% of blood, and is comprised of blood serum and fibrinogen. It involves in transportation of material, exchange of gas, blood coagulation, or immunity, and plays significant roles to control internal circumstances by adjusting osmotic pressure, hydrogen-ion concentration, etc. The “blood plasma” to be measured by the present invention is not particularly limited, but includes those from blood of mammals such as humans, rats, mice, dogs, monkeys, etc. The production method thereof is also not particularly limited, but may include conventional methods. The blood plasma is preferably prepared by drawing blood, placing the blood into a plastic tube containing an anticoagulant (e.g., heparin sodium, EDTA-2Na, EDTA-2K, etc.), cooling the mixture with ice, and centrifuging the mixture at 4° C. to collect blood plasma.

Gas chromatography/mass spectrometry (GC/MS method) is useful for qualitative or quantitative analysis of organic compounds (particularly, low-molecular-weight components) by an analysis equipment comprising a gas chromatograph (GC) and a mass spectometer (MS) linked together. In the method, qualitative analysis of components is performed by measuring MS spectrum of a single component separated by GC, and quantitative analysis is performed by measuring ion intensity detected by MS. Examples of “gas chromatography/mass spectrometry (GC/MS method)” in the present invention is not particularly limited, but include those generally used for identification, quantitative analysis, etc. of substances, preferably those using electron-impact ionization (EI), those using chemical ionization (CI), and the like (for example, the above Non-Patent Documents 10, 11 and 12).

The method of the present invention comprises extracting acetic acid in blood plasma with methyl-tert-butyl ether (MTBE).

For extracting acetic acid in blood plasma, hexane, diethyl ether, etc. have been conventionally used. It is, however, necessary to carefully handle diethyl ether because of its low boiling point. In addition, there are some problems with these solvents as follows: hexane provides low extraction efficiency of acetic acid; the extraction with diethyl ether requires the addition of a certain amount of hydrochloric acid beforehand (for example, 0.025 to 0.125 mL of concentrated hydrochloric acid (12N) as hydrochloric acid is required relative to 1 mL of a solution after deproteinization). These solvents, therefore, make the operating procedures complicated.

In contrast, MTBE has a boiling point higher than diethyl ether and provides higher extraction efficiency of acetic acid than diethyl ether or hexane, and thus does not cause the above problems with conventional extracting solvents.

In the present invention, the amount of MTBE to be used can be determined based on the amount of blood plasma, and it is generally 0.004 to 40 mL, preferably 0.04 to 4 mL, relative to 0.4 mL of blood plasma.

In general, the above extraction with MTBE is carried out as follows. Firstly, a certain amount of MTBE is mixed to blood plasma, and then the mixture is stirred for a certain time. The stirring time and temperature are not particularly limited, but the stirring time may include, for example, 30 seconds to 1 minute and the temperature may include, for example, 15 to 30° C. After that, the mixture is centrifuged to remove a precipitate and to collect a supernatant. The centrifugation may be carried out, for example, at 10000 rpm at 4 to 20° C. for 5 to 10 minutes.

As a result of the above procedures, acetic acid in blood plasma is extracted into a supernatant (MTBE).

When diethyl ether, which is conventionally used as an extracting solvent, is used for the extraction of acetic acid, it is necessary to add a certain amount of hydrochloric acid to blood plasma beforehand (for example, 0.025 to 0.125 mL of concentrated hydrochloric acid (12N) as hydrochloric acid is required relative to 1 mL of a solution after protein removal). This is because an aqueous solution needs to be acidified when using diethyl ether. From the present comparative investigation, it has, however, been found that the MTBE as an extracting solvent can provide higher extraction efficiency than diethyl ether without requiring the addition of hydrochloric acid. The method of the present invention is thus simpler and achieves a higher yield compared to conventional methods.

The method of the present invention may further comprise treating blood plasma with a deproteinizing agent. Examples of the deproteinizing agent to be used in the method of the present invention include sulfosalicylic acid, perchloric acid, metaphosphoric acid, and the like, preferably sulfosalicylic acid.

By the treatment of blood plasma with the deproteinizing agent, proteins in blood plasma are coagulated and suspended in blood plasma. As a result, proteins in blood plasma are substantially removed (deproteinizing step). Conventionally, proteins suspended in blood plasma have been removed as a precipitate by centrifugation and only supernatant has been used for the measurements by GC/MS methods and the like, because such proteins have been considered to cause adverse effects on the measurements.

The present inventors have, surprisingly, found that the product of deproteinizationcan be directly, without centrifugation, subjected to extraction of acetic acid with MTBE. Namely, no centrifugation after the treatment with the deproteinizing agent is necessary for the present invention, and the number of the operating procedures is thus reduced. If 100 samples are measured by the method of the present invention, it is possible to shorten the time for operating procedures by about 60 minutes compared to conventional methods.

The amount of the deproteinizing agent to be used in the present invention can be determined based on the amount of blood plasma. For example, when 10% sulfosalicylic acid is used as the deproteinizing agent, the amount thereof may be generally 0.001 to 10 mL, preferably 0.01 to 1 mL, relative to 0.4 mL of blood plasma. The concentration of the deproteinizing agent can be arbitrarily varied, thereby changing the yield.

The above treatment with the deproteinizing agent is generally carried out as follows. Firstly, a certain amount of the deproteinizing agent is mixed to blood plasma, and the mixture is stirred for a certain time. The stirring time and temperature are not particularly limited, but the stirring time may include, for example, 30 seconds to 1 minute and the temperature may include, for example, 15 to 30° C. As a result, proteins in blood plasma are coagulated to give a suspension.

In conventional methods, the suspension is subjected to centrifugation to remove suspended proteins as a precipitate. In contrast, in the method of the present invention, it is possible to subject the suspension from the treatment of the deproteinization to the next step (in general, MTBE extraction) without centrifugation

In the method of the present invention, the treatment with the deproteinizing agent is generally performed before the extraction of acetic acid in blood plasma with MTBE. By this step, the interference by proteins can be prevented in the extraction with MTBE.

The method of the present invention may further comprise adding a stable isotope labeled acetic acid to blood plasma. The stable isotope refers to isotopes not releasing radiation and existing semipermanently without changing the abundance thereof. Examples of the stable isotope to be used for labeling include, in addition to stable isotopes of carbon (¹²C and ¹³C), stable isotopes of hydrogen (¹H and ²H(D)), stable isotopes of oxygen (¹⁶O, ¹⁷O, and ¹⁸O), and the like. The stable isotope labeled acetic acid can be used as an internal standard, thereby correcting the variations of the results depending on the measuring operation.

Examples of the “stable isotope labeled acetic acid” in the present invention include sodium acetate-1-¹³C, sodium acetate-2-¹³C, sodium acetate-¹³C₂, sodium acetate-d₃, sodium acetate-¹⁸O₂, sodium acetate-1-¹³C,d₃ and sodium acetate-2-¹³C,d₃, and the like, preferably sodium acetate-2-¹³C,d₃, and the like. They are commercially available, and can be purchased from, for example, ISOTEC, Cambridge Isotope Laboratories, etc.

In the method of the present invention, the addition of the stable isotope labeled acetic acid to blood plasma is generally carried out before the protein removal or MTBE extraction because the internal standard is used for correcting the variations depending on the measuring operation.

In the method of the present invention, a derivatizing reagent is generally added to the sample obtained by the above each step to derivatize acetic acid in the sample, and then the concentration of acetic acid in the sample is measured by the GC/MS method. The derivatizing reagent to be used in the present invention is not particularly limited, but includes, for example, N-methyl-N-(tert-butyldimethylsilyl) trifluoroacetamide (MTBSTFA), t-butyldimethylsilylimidazole (TBDMS), and the like. They are commercially available, and can be purchased from, for example, Sigma-Aldrich, Fulka, Pierce, etc.

The present invention further provides a kit for measuring the concentration of acetic acid in blood plasma, which can be used for the above method of the present invention for measuring the concentration of acetic acid in blood plasma by a GC/MS method.

The kit of the present invention may comprise various reagents, which can be used for the method of the present invention, particularly the kit may one or more reagents such as MTBE, deproteinizing agent, stable isotope labeled acetic acid, derivatizing reagent for GC/MS method, sodium acetate, etc.

### EXAMPLES

### Example 1

### 1. Preparation of Acetic Acid Standard Solutions

Sodium acetate (82 mg, Lot No. MKBD4171V, manufactured by SIGMA ALDRICH) was precisely weighted with an electric balance (AX205DR, manufactured by Mettler Toledo) and dissolved and made up to exactly 10 mL with purified water to prepare a 100 mmol/L solution (S). Further, as shown in Table 1, standard solutions (W1 to W8) were prepared by using a measuring flask before use.

TABLE 1

| | Concentration | | Preparation |
|---|---|---|---|
| W1 | 40 | mmol/L | To S (4 mL) was added purified |
| | | | water to make exactly 10 mL. |
| W2 | 10 | mmol/L | To S (1 mL) was added purified |
| | | | water to make exactly 10 mL. |
| W3 | 1 | mmol/L | To W2 (1 mL) was added purified |
| | | | water to make exactly 10 mL. |
| W4 | 500 | μmol/L | To W3 (5 mL) was added purified |
| | | | water to make exactly 10 mL. |
| W5 | 250 | μmol/L | To W4 (5 mL) was added purified |
| | | | water to make exactly 10 mL. |
| W6 | 100 | μmol/L | To W5 (4 mL) was added purified |
| | | | water to make exactly 10 mL. |
| W7 | 50 | μmol/L | To W6 (5 mL) was added purified |
| | | | water to make exactly 10 mL. |
| W8 | 20 | μmol/L | To W7 (4 mL) was added purified |
| | | | water to make exactly 10 mL. |

### 2. Preparation of Internal Standard Solution

Stable isotope labeled sodium acetate (8.6 mg, Lot No. EK1873: sodium acetate-2-¹³C, d₃, manufactured by ISOTEC) was precisely weighted and dissolved and made up to exactly 10 mL with purified water to prepare a 10 mmol/L solution (IS). Next, 10 mmol/L solution (IS) (2 mL) was taken into a measuring flask, made up to exactly 10 mL with purified water to prepare a 2 mmol/L solution (IS-W1).

### 3. Samples for Calibration Curve

As samples for calibration curve, the standard solutions W3 to 8 were used.

### 4. Pretreatment Method and Measurement Method for Samples

Into a polypropylene tube, each sample (400 μL, samples for calibration curve and human blood plasma) was taken, and the internal standard solution IS-W1 (100 μL) was added thereto. Further, 10% sulfosalicylic acid (special grade, manufactured by Wako Pure Chemical Industries) solution (100 μL) was added thereto and stirred. To this solution was added MTBE (400 μL, Lot No. EPF0734, manufactured by Wako Pure Chemical Industries) and stirred for 1 minute. After that, the mixture was subjected to centrifugation (RX-200, manufactured by TOMY SEIKO) at 10000 rpm at 4° C. for 5 minutes, and the supernatant (200 μL) was taken into another glass vial. Then, to the supernatant was added N-methyl-N-(tert-butyldimethylsilyl)trifluoroacetamide (MTBSTFA) (5 to 10 μL, Lot No. BCBC7878, manufactured by ALDRICH) and heated with Block Heater (DTU-2B, manufactured by TAITEC) at 60° C. for 1 to 2 hours. After that, the resultant solution was measured by using GC/MS (JMS-AMII150, manufactured by JEOL) in the following conditions.

[GC/MS measurement condition]
Column: DB-5MS (length 30 m, internal diameter 0.25 mm, film thickness 0.25 μm, manufactured by Agilent)
Injection method and: Split injection (50:1), 1 μL
injection amount
Temperature of: 200° C.
injection port
Carrier gas and flow: Helium and 1 mL/min
rate
Temperature rising: STEP1 40° C. (0.1 min) to 70° C. (3
condition min) at 5° C./min STEP2 70° C. to 280° C. (4 min) at 70° C./min
Ionization method: EI method
Ionization voltage: 70 eV, 300 μA
and Electric current
Monitoring ions: m/z 117 (standard) m/z 121 (internal standard)
Ion source: 250° C.
temperature
Interface: 250° C.
temperature

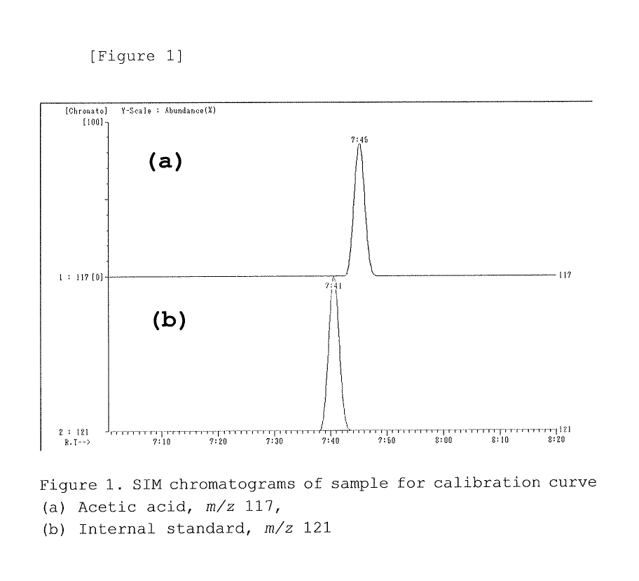

FIG. 1 shows SIM chromatograms of acetic acid and the internal standard in a sample for calibration curve according to the method of Example 1. Acetic acid and the internal standard were separated and eluted in the vicinity of 7.45 and 7.41 min, respectively.

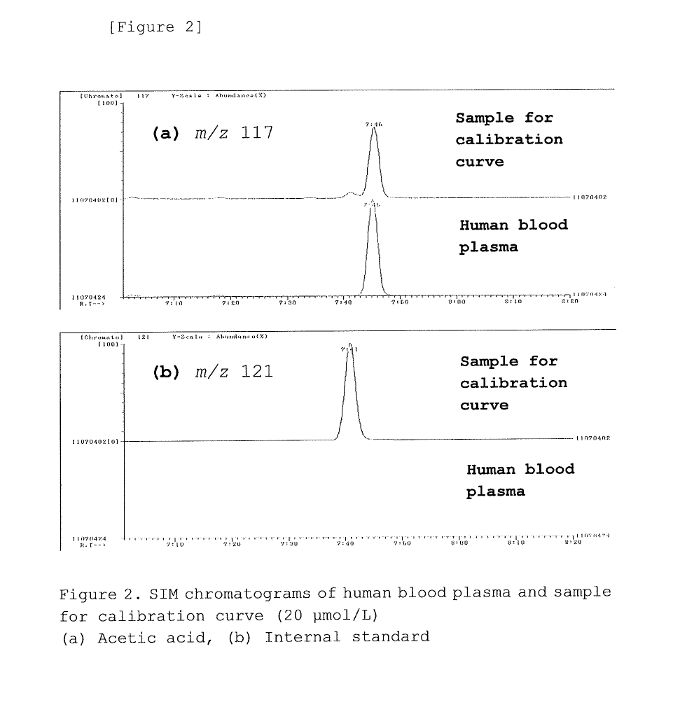

Samples for calibration curve together with human blood plasma were pretreated and measured in the same method as that of Example 1. Both mass chromatograms were compared and assessed for the presence or absence of interference peaks at the elution position of the internal standard. FIG. 2 shows SIM chromatograms comparing human blood plasma with a sample for calibration curve (20 μmol/L). There was no interference peak derived from blood plasma at the elution position of the internal standard.

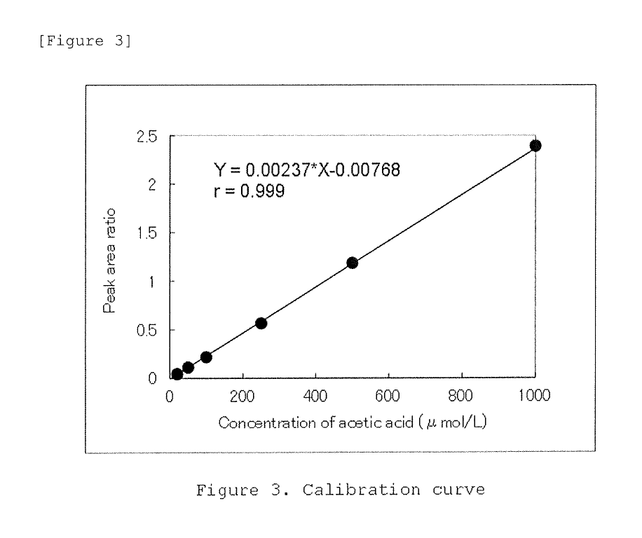

Samples for calibration curve (standard solutions W3 to 8) were pretreated and measured in the same method as that of Example 1. The relative error (% RE) in each concentration in the calibration curve and the linearity of the calibration curve were examined. FIG. 3 shows the calibration curve obtained. In the range of the concentration of 20 to 1000 μmol/L, the linearity of the calibration curve showed 0.999 of correlation coefficient (r), and % RE of each concentration of the calibration curve was from −5.4 to 7.5%.

Next, the peak areas of acetic acid and a stable isotope labeled acetic acid with or without the hydrochloric acid pretreatment were compared. The amount of each sample added is shown below.

### Comparative Example 1

### Examination of Standard Solutions

| | |
|---|---|
| 1 mmol/L Na acetate standard solution | 400 μL |
| 2 mmol/L stable isotope labeled Na acetate | 100 μL |
| 10% sulfosalicylic acid | 100 μL |
| MTBE or diethyl ether (DEE) | 400 μL |
| 6N HCl or H₂O | 50 μL |

The peak areas of the above samples were obtained according to the method described in Example 1. The results are shown in Table 2.

### Comparative Example 2

### Examination of Blood Plasma Samples

| | |
|---|---|
| Blood plasma sample | 400 μL |
| 2 mmol/L stable isotope labeled Na acetate | 100 μL |
| 10% sulfosalicylic acid | 100 μL |
| MTBE or diethyl ether (DEE) | 400 μL |
| 6N HCl or H₂O | 50 μL |

The peak areas of the above samples were obtained according to the method described in Example 1. The results are shown in Table 3.

TABLE 2
Examination of standard solutions

| Addition | | Stable isotope | | | | | |
|---|---|---|---|---|---|---|---|
| of hydro- | Extract- | labeled acetic acid | | | Acetic acid | | |
| chloric | ing | Peak | Mean | | Peak | Mean | |
| acid | solvent | area | value | % | area | value | % |
| No | MTBE | 57861 | 57861 | 100 | 130888 | 131259 | 100 |
| | | 58202 | | | 131797 | | |
| | | 58022 | | | 131091 | | |
| | DEE | 41864 | 45925 | 79 | 92109 | 100964 | 77 |
| | | 47753 | | | 105195 | | |
| | | 48159 | | | 105535 | | |
| Yes | MTBE | 2185 | 2060 | 4 | 4819 | 4497 | 3 |
| | | 2309 | | | 4919 | | |
| | | 1685 | | | 3752 | | |
| | DEE | 45182 | 41789 | 72 | 96832 | 89527 | 68 |
| | | 41537 | | | 88943 | | |
| | | 38649 | | | 82806 | | |

TABLE 3
Examination of blood plasma

| Addition | | Stable isotope | | | | | |
|---|---|---|---|---|---|---|---|
| of hydro- | Extract- | labeled acetic acid | | | Acetic acid | | |
| chloric | ing | Peak | Mean | | Peak | Mean | |
| acid | solvent | area | value | % | area | value | % |
| No | MTBE | 55061 | 59219 | 100 | 10099 | 10838 | 100 |
| | | 63377 | | | 11576 | | |
| | DEE | 53200 | 58021 | 98 | 9793 | 10576 | 98 |
| | | 62841 | | | 11358 | | |
| Yes | MTBE | 4601 | 4033 | 7 | 1309 | 1223 | 11 |
| | | 3465 | | | 1136 | | |
| | DEE | 48582 | 45474 | 77 | 9169 | 8530 | 79 |
| | | 42366 | | | 7891 | | |

As shown in Tables 2 and 3, the extraction with MTBE without the addition of hydrochloric acid gave the best result in extraction efficiency in both standard solutions and blood plasma.

### Example 2

### 1. Preparation of Standard Solutions

Solutions W1 to 8 obtained in “1. Preparation of acetic acid standard solutions” of Example 1 were used.

### 2. Preparation of Internal Standard Solution

Solution IS-W1 obtained in “2. Preparation of internal standard solution” of Example 1 was used.

### 3. Samples for Calibration Curve

As samples for calibration curve, the standard solutions W3 to 8 were used.

### 4. Samples of Acetic Acid-Added Blood Plasma

Standard solution S (180 μL) was taken into a 10 mL measuring flask and diluted to 10 mL with human blood plasma to prepare an acetic acid (1800 μmol/L) added blood plasma (P1). Further, as shown below, acetic acid added blood plasmas (P2 to 5) were prepared by using commercial human blood plasma.

| | Added concentration | Preparation |
|---|---|---|
| P2 | 900 μmol/L | To P1 (4 mL) was added human |
| | | blood plasma (4 mL). |
| P3 | 450 μmol/L | To P2 (4 mL) was added human |
| | | blood plasma (4 mL). |
| P4 | 150 μmol/L | To P3 (2 mL) was added human |
| | | blood plasma (4 mL). |
| P5 | 50 μmol/L | To P4 (2 mL) was added human |
| | | blood plasma (4 mL). |

### 5. Pretreatment Method and Measurement Method for Samples

Samples were pretreated in the same method as that of “4. Pretreatment method and measurement method for samples” of Example 1, and then measured by GC/MS (5975C GCMSD, manufactured by Agilent) in the following conditions.

### [GC/MS Analysis Conditions]

Column: HP-5MS (length 30 m, internal diameter 0.25 mm, film thickness 0.25 μm, manufactured by Agilent)
Injection method and: Split injection (50:1), 1 μL
injection volume
Temperature of: 200° C.
injection port
Carrier gas and flow: Helium and 1 mL/min
rate
Temperature rising: STEP 1 50° C. (0.1 min) to 70° C. (3
condition min) at 6° C./min STEP 2 70° C. to 300° C. (2 min) at 70° C./min
Ionization method: EI method
Ionization voltage: 70 eV
Monitoring ions: m/z 117 (standard): m/z 121 (internal standard)
Ion source: 230° C.
temperature
Quadrupole: 150° C.
temperature
AUX temperature: 280° C.

### 7. Calibration Curve

Calibration curve used in Example 1 was used.

### Comparative Example 3

### Enzyme Method (Acetyl CoA Synthase Method)

### 1. Samples for Calibration Curve

Solutions W2 to 8 obtained in Example 2 were used.

### 2. Samples of Acetic Acid Added Blood Plasma

Solutions P1 to 5 obtained in Example 2 were used.

### 3. Samples of Blood Plasma from Dialysis Patient

Blood plasma from patient (n=45) being treated with a dialysis fluid containing acetic acid at 2 and 4 hours after the dialysis initiation were used as the samples.

### 4. Measurement by Kit for Measuring Acetic Acid (F-Kit Acetic Acid)

Each sample (200 μL, samples for calibration curve, human blood plasma, samples of acetic acid-added blood plasma and samples of blood plasma from dialysis patient) was taken into a cuvette, and purified water (800 μL) was added thereto. The measurement was performed in accordance with the method described in the package insert of F-kit acetic acid (Lot/Ch.-B.: 12670700, J. K. international) except for using a half amount of the reagent solution attached to the kit.

[Measurement wavelength]

Wavelength: 340 nm

### 5. Calibration Curve

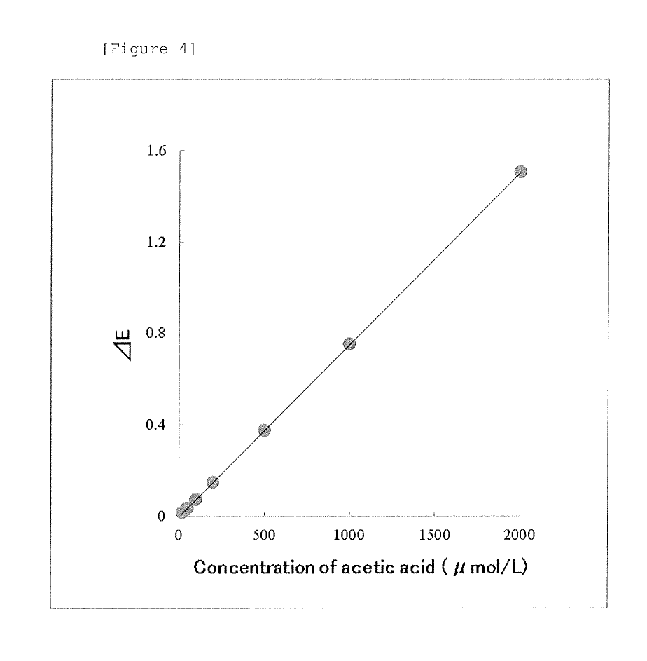

From ΔE of samples for calibration curve (W2 to 8, n=1), calibration curve (Y=aX+b, Y: ΔE, X: concentration μmol/L) was prepared by using least-squares method. No weighting of calibration curve was carried out. The resultant calibration curve is shown in FIG. 4. The range of quantitative analysis was from 20 to 2000 μmol/L.

### Comparison of Results Between Example 2 and Comparative Example 3

### 1. Calculation Method of Concentration

The measured value of each sample was calculated by applying the peak area ratio or ΔE to the calibration curve, and rounding off to one decimal place.

### 2. Calculation Method of Relative Error (RE)

RE was calculated according to the following equation:

$$RE ( \% ) = \frac{Measured Value - Theoretical Value}{Theoretical Value} \times 100$$

### 3. Calibration Curve

The measurement results of samples for calibration curve by the GC/MS method (Example 2) and the enzyme method (Comparative Example 3) are shown in the following Tables 4 and 5. Both methods produce a good result.

TABLE 4
GC/MS Method

| Concentration (μmol/L) | | | | | | | | | | | | |
|---|---|---|---|---|---|---|---|---|---|---|---|---|
| | RE | | RE | | RE | | RE | | RE | | RE | |
| 20 | (%) | 50 | (%) | 100 | (%) | 200 | (%) | 500 | (%) | 1000 | (%) | r |
| 20.4 | 2.2 | 48.3 | −3.4 | 95.9 | −4.1 | 196.0 | −2.0 | 512.3 | 2.5 | 1048.4 | 4.8 | 0.9990 |

r: correlation coefficient

TABLE 5
Enzyme Method

| Concentration (μmol/L) | | | | | | | | | | | | | | |
|---|---|---|---|---|---|---|---|---|---|---|---|---|---|---|
| | RE | | RE | | RE | | RE | | RE | | RE | | RE | |
| 20 | (%) | 50 | (%) | 100 | (%) | 200 | (%) | 500 | (%) | 1000 | (%) | 2000 | (%) | r |
| 19.2 | −4.1 | 49.6 | −0.8 | 98.5 | −1.5 | 199.2 | −0.4 | 501.0 | 0.2 | 998.8 | −0.1 | 1999.3 | 0 | 0.9999 |
| 20.9 | 4.7 | 45.7 | −8.6 | 95.2 | −4.8 | 196.3 | −1.9 | 499.5 | −0.1 | 998.1 | −0.2 | 1997.0 | −0.1 | 0.9999 |

r: correlation coefficient

### 4. Samples of Blood Plasma and Acetic Acid-Added Blood Plasma

The measurement results of blood plasma and acetic acid-added blood plasma by the GC/MS method (Example 2) and the enzyme method (Comparative Example 3) are shown in the following Tables 6 and 7. In the GC/MS method, the recovery rate was a good value, 92% or more, at every concentration of the added acetic acid. In the enzyme method, the recovery rate was 90% or less at 50 and 150 μmol/L of the concentration of the added acetic acid.

Comparison of the measured values in both methods is shown in the following Table 8. For every sample, the GC/MS method gave higher value than the enzyme method. Both measured values tended to be closer with increasing the concentration of the added acetic acid.

TABLE 6
GC/MS Method

| | Concentra- | | | | | |
|---|---|---|---|---|---|---|
| | tion of added | Measured | Mean | | | |
| | acetic acid | Value | value | | | Recovery |
| | (μmol/L) | (μmol/L) | (μmol/L) | S.D. | C.V. | Rate |
| Human | 0 | 34.3 | 35.3 | 0.9 | 2.6 | |
| blood | | 36.0 | | | | |
| plasma | | 35.8 | | | | |
| P5 | 50 | 84.2 | 83.3 | 1.6 | 2.0 | 96.0 |
| | | 81.4 | | | | |
| | | 84.3 | | | | |
| P4 | 150 | 175.9 | 174.5 | 5.0 | 2.8 | 92.8 |
| | | 178.6 | | | | |
| | | 169.0 | | | | |
| P3 | 450 | 457.0 | 470.8 | 12.2 | 2.6 | 96.8 |
| | | 475.9 | | | | |
| | | 479.6 | | | | |
| P2 | 900 | 937.3 | 925.8 | 22.6 | 2.4 | 98.9 |
| | | 899.8 | | | | |
| | | 940.3 | | | | |
| P1 | 1800 | 1781.3 | 1797.8 | 14.5 | 0.8 | 97.9 |
| | | 1803.8 | | | | |
| | | 1808.5 | | | | |

TABLE 7
Enzyme Method

| | Concentra- | | | | | |
|---|---|---|---|---|---|---|
| | tion of added | Measured | Mean | | | |
| | acetic acid | value | value | | | Recovery |
| | (μmol/L) | (μmol/L) | (μmol/L) | S.D. | C.V. | rate |
| Human | 0 | 1.9 | 1.9 | 0.0 | 1.2 | |
| blood | | 1.9 | | | | |
| plasma | | 1.9 | | | | |
| P5 | 50 | 34.4 | 41.0 | 6.6 | 16.2 | 78.2 |
| | | 40.9 | | | | |
| | | 47.7 | | | | |
| P4 | 150 | 132.8 | 132.1 | 5.0 | 3.7 | 86.9 |
| | | 136.8 | | | | |
| | | 126.9 | | | | |
| P3 | 450 | 409.4 | 407.2 | 2.0 | 0.5 | 90.1 |
| | | 407.0 | | | | |
| | | 405.3 | | | | |
| P2 | 900 | 835.7 | 835.9 | 1.8 | 0.2 | 92.7 |
| | | 837.9 | | | | |
| | | 834.3 | | | | |
| P1 | 1800 | 1713.9 | 1711.9 | 9.6 | 0.6 | 95.0 |
| | | 1720.4 | | | | |
| | | 1701.5 | | | | |

TABLE 8

| | Concentration of | GC/MS | Enzyme | Values relative to |
|---|---|---|---|---|
| | added acetic acid | Method | method | values by GC/MS |
| | (μmol/L) | (μmol/L) | (μmol/L) | method as 100 |
| Human | 0 | 35.3 | 1.9 | 5 |
| blood | | | | |
| plasma | | | | |
| P5 | 50 | 83.3 | 41.0 | 49 |
| P4 | 150 | 174.5 | 132.1 | 76 |
| P3 | 450 | 470.8 | 407.2 | 86 |
| P2 | 900 | 925.8 | 835.9 | 90 |
| P1 | 1800 | 1797.8 | 1711.9 | 95 |

### 5. Samples of Blood Plasma from Dialysis Patient

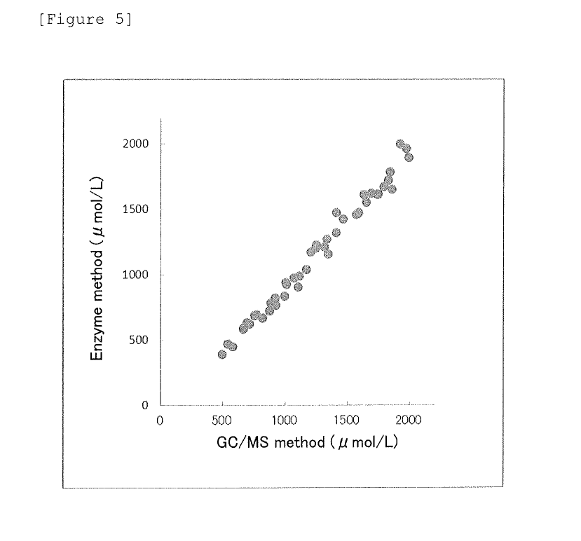

Table 9 shows measured values by the enzyme method (Comparative Example 3) for blood plasma of dialysis patient (n=45) being treated with an acetic acid-containing dialysis fluid, and measured values by the GC/MS method (Example 2) for the same samples. In 43 cases among 45 cases, measured values by the GC/MS method were higher than those by the enzyme method. FIG. 5 shows a plot where the abscissa axis is the measured values obtained by the GC/MS method and the vertical axis is the measured values obtained by the enzyme method. There was a good correlation (r=0.9922) between the measured values obtained by both methods.

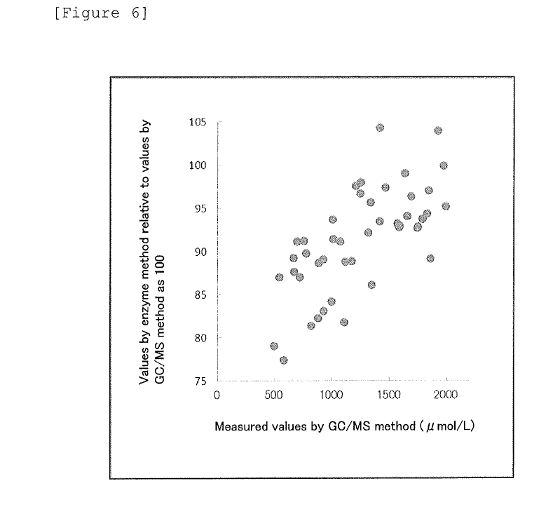

FIG. 6 shows a plot where the abscissa axis is the values obtained by GC/MS method, and the vatical axis values obtained by the enzyme method relative to the values obtained by the GC/MS method as 100. Both measured values tended to be closer with increasing the measured value by the GC/MS method.

TABLE 9

| | |
|---|---|
| | Measured value |
| Dialysis | (μmol/L) |

| Patient | time | Enzyme | GC/MS |
|---|---|---|---|
| No. | (hr) | method | method |
| 1 | 2 | 472.9 | 543.4 |
| 2 | 2 | 450.1 | 581.5 |
| 3 | 2 | 393.9 | 498.5 |
| 4 | 2 | 634.0 | 695.9 |
| 5 | 2 | 585.8 | 668.4 |
| 6 | 2 | 592.8 | 664.3 |
| 7 | 2 | 693.8 | 773.0 |
| 8 | 2 | 624.8 | 717.9 |
| 9 | 2 | 688.8 | 755.3 |
| 10 | 2 | 783.8 | 884.3 |
| 11 | 2 | 721.9 | 877.5 |
| 12 | 2 | 667.3 | 820.0 |
| 13 | 2 | 770.2 | 927.0 |
| 14 | 2 | 838.9 | 996.5 |
| 15 | 2 | 821.5 | 922.2 |
| 16 | 2 | 975.7 | 1070.8 |
| 17 | 2 | 925.2 | 1012.4 |
| 18 | 2 | 942.8 | 1006.5 |
| 19 | 2 | 906.2 | 1107.5 |
| 20 | 2 | 1040.1 | 1170.1 |
| 21 | 2 | 991.9 | 1116.7 |
| 22 | 2 | 1176.1 | 1205.1 |
| 23 | 2 | 1204.7 | 1245.5 |
| 24 | 2 | 1226.4 | 1251.2 |
| 25 | 2 | 1275.1 | 1333.1 |
| 26 | 2 | 1213.0 | 1315.8 |
| 27 | 2 | 1159.9 | 1347.1 |
| 28 | 2 | 1427.5 | 1466.2 |
| 29 | 2 | 1320.4 | 1412.2 |
| 30 | 2 | 1473.9 | 1412.3 |
| 31 | 2 | 1477.0 | 1589.4 |
| 32 | 2 | 1466.9 | 1581.0 |
| 33 | 2 | 1462.2 | 1569.1 |
| 34 | 2 | 1626.4 | 1687.9 |
| 35 | 2 | 1615.5 | 1631.5 |
| 36 | 2 | 1555.3 | 1652.4 |
| 37 | 4 | 1676.6 | 1788.7 |
| 38 | 2 | 1614.7 | 1741.3 |
| 39 | 4 | 1620.3 | 1745.5 |
| 40 | 2 | 1788.2 | 1842.1 |
| 41 | 2 | 1724.1 | 1826.6 |
| 42 | 4 | 1653.5 | 1854.9 |
| 43 | 4 | 1896.4 | 1991.5 |
| 44 | 2 | 1966.7 | 1968.4 |
| 45 | 2 | 1998.6 | 1922.0 |

As described above, the GC/MS method (Example 2) has superior sensitivity and specificity compared to the enzyme method (Comparative Example 3). The GC/MS method is thus considered to be useful for measuring the concentration of acetic acid in blood plasma.

Enzyme methods have been reported to be sensitive to interfering substances in blood plasma (see Bergmeyer H U and Mollering H: In: Methods of Enzymatic Analysis (ed by Bergmeyrer H U, et al), 3^{rd} ed, vol VI, p. 628-645, W2-einheim, Deerfield Beach, Fla., Verlag Chimemine, 184. (1985)). The above results implied this matter.

1. A method for measuring the concentration of acetic acid in blood plasma by using a gas chromatography/mass spectrometry (GC/MS method), which comprises extracting acetic acid in blood plasma with methyl-tert-butyl ether (MTBE).

2. The method according to claim 1, which does not comprise adding hydrochloric acid before extracting.

3. The method according to claim 1, which further comprises treating blood plasma with a deproteinizing agent.

4. The method according to claim 1, wherein the deproteinizing agent includes sulfosalicylic acid.

5. The method according to claim 3, which comprises extracting acetic acid in blood plasma with MTBE, after treating blood plasma with a deproteinizing agent and not centrifuging.

6. The method according to claim 1, which further comprises adding a stable isotope labeled acetic acid to blood plasma.

7. The method according to claim 6, wherein the stable isotope labeled acetic acid is selected from sodium acetate-1-¹³C, sodium acetate-2-¹³C, sodium acetate-¹³ C₂, sodium acetate-d₃, sodium acetate-¹⁸O₂, sodium acetate-1-¹³C,d₃ and sodium acetate-2-¹³C,d₃.

8. The method according to claim 1, wherein the GC/MS method includes electron impact ionization.

9. A kit for measuring the concentration of acetic acid in blood plasma by using a GC/MS method, comprising MTBE.

10. The kit according to claim 9, which further comprises a deproteinizing agent.

11. The kit according to claim 10, wherein the deproteinizing agent includes sulfosalicylic acid.

12. The kit according to claim 9, which further comprises a stable isotope labeled acetic acid.

13. The kit according to claim 12, wherein the stable isotope labeled acetic acid is selected from sodium acetate-1-¹³C, sodium acetate-2-¹³C, sodium acetate-¹³C₂, sodium acetate-d₃, sodium acetate-¹⁸O₂, sodium acetate-1-¹³C,d₃ and sodium acetate-2-¹³C,d₃.

14. The kit according to claim 9, which further comprises a derivatizing reagent.

15. The kit according to claim 14, wherein the derivatizing reagent is N-methyl-N-(tert-butyldimethylsilyl)trifluoroacetamide.
